(19) United States
(12) Patent Application Publication
(10) Pub. No.: US 2026/0188465 A1
(43) Pub. Date: Jul. 2, 2026

(54) METHOD AND SYSTEM FOR RECOMMENDING PERSONAL LIFESTYLE AND DIET MODIFICATIONS BASED ON MEDICAL AND GENETIC INPUTS

(71) Applicant: HealthSpans.com LLC, Fort Lauderdale, FL (US)

(72) Inventors: Michael YUZ, Fort Lauderdale, FL (US); Sharon YUZ, Fort Lauderdale, FL (US); Jonathan YUZ, Fort Lauderdale, FL (US); Bellen YUZ, Fort Lauderdale, FL (US); Nancy KANEVSKI, Fort Lauderdale, FL (US); Marcus THILEN, Fort Lauderdale, FL (US); Elli YUZ, Fort Lauderdale, FL (US)

(21) Appl. No.: 19/438,142

(22) Filed: Dec. 31, 2025

Related U.S. Application Data
(60) Provisional application No. 63/741,221, filed on Jan. 2, 2025.

(51) Int. Cl. G16H 20/60; G09B 5/02

(52) U.S. Cl. CPC G16H 20/60; G09B 5/02

(57) ABSTRACT

A computer-based method and system for extending a user's healthspan and decreasing the user's rate of aging is disclosed. In one embodiment, the method includes receiving user information including identification information, medical information, and at least one of biometric information, lab information, medical imaging reports and images of the user, processing and analyzing the received information using at least one of AI technology, machine learning and algorithms, and providing accurate health, wellness and longevity information and recommendations to the user.

### CROSS-REFERENCE TO RELATED APPLICATION

This application claims priority to U.S. Provisional Application No. 63/741,221, filed Jan. 2, 2025, entitled “METHOD AND SYSTEM FOR RECOMMENDING PERSONAL LIFESTYLE AND DIET MODIFICATIONS BASED ON MEDICAL AND GENETIC INPUTS,” the entirety of which is incorporated herein by reference.

### FIELD OF INVENTION

This disclosure relates to a method and system for assisting patients in improving their diet, prevent or control diseases, extend their healthy lifespans (“healthspans”) and reduce their rate of aging.

### BACKGROUND

Research and applications directed at health, lifestyle and longevity, i.e., “the longevity market” is one of the world's fastest growing industries. Analysts predict the global longevity industry will be worth at least $600 billion by 2025. The market for complementary and alternative medicine for anti-aging and longevity is expected to grow from $63.11 billion in 2023 to $366.58 billion by 2032. In addition, people over 65 are projected to spend almost $15 trillion a year by 2030, up from $8.7 trillion in 2020 in this fast-growing market.

The exponential growth in the longevity industry is based on several factors. These include recent scientific advances; numerous recent high profile public relations and significant interest from consumers and media; demographics—the baby boomer generation is aging, and they own a large portion of the country's wealth; spending habits-as people age, they spend more on wellness, treatment, senior care, and discretionary spending; and savings—in developing countries, seniors are accumulating higher savings than previous generations.

Personalized medicine, also known as precision medicine, is a medical and health concept that tailors medical care and life choices to a patient's individual characteristics. Longevity medicine has not yet been officially defined by a central medical body, but expert recommendations suggest that the goal of longevity medicine is to establish and restore the biological age of an individual at each specific point of time to the biological age of the optimal individual performance. The result is elimination or reduction of risks of age-related and overall morbidity. Therefore, the main focus of longevity medicine is to prolong a life lived in good health, both physically and mentally, extension of the healthy lifespan and not solely the health (simple prevention) or solely the lifespan (reactive medicine/sick care).

Many experts today recognize that both nature and nurture interact with each other in important ways to determine a person's morbidity and mortality. Nature refers to fixed genetic influence, whereas nurture refers to how their environment (including diet, exercise, environment, relationships, emotional factors, purpose, relationships and stress level, etc.) impacts human organisms. While both factors are important, as per recent research, genetics contributes only between 20% and 25% to how healthy we are and how long we live, and 75% to 80% is due to other/external factors.

Chronic diseases can decrease life expectancy. Many chronic diseases have considerable impact on life expectancy and health care costs. The expected reduction in average life expectancy ranges from 0.3 years for a 65-year-old with hypertension to more than three years for an average person with stroke or diabetes. Chronic diseases are the leading cause of death and disability in the United States. However, they can often be prevented or managed effectively through diet, behavior change, stress management, medical management and monitoring.

A Johns Hopkins study found that the more chronic conditions a person has the shorter their life expectancy. For example, a 67-year-old with five chronic conditions might live 7.7 years fewer than someone with no chronic conditions. One study estimated that people with stroke and diabetes might have a minimum reduction of 33% in survival. Another study found that people with osteoporosis might lose more than four years of life expectancy. After age 50, one in two women and one in four men will experience a fracture related to osteoporosis. Broken hips are especially serious and can increase the risk of death.

As per the Johns Hopkins study, life expectancy in U.S. is falling in the past several due to surges in chronic illnesses. In 1900, U.S. life expectancy was 47 years, and by 2019 it hit 79. But in 2020, life expectancy fell to 77 and dropped further to 76.4 in 2021, according to a report from the Centers for Disease Control and Prevention.

Many people are not aware that they have certain chronic diseases and conditions, in preclinical stages or predisposed in developing them.

Diet is thought to be one of the most important external factors determining human longevity but also one of the most complex to control. In one dramatic example, the results of one meta-study examined telomere shortening as associated with increased mortality risk in the general population. (Telomere is a region of repetitive DNA sequences at the end of a chromosome.) Several studies show that the Mediterranean Diet and the absence of cigarette smoking are correlated with longer telomere length, while the relationship with physical activity is not clearly established. It has been proven that a poor diet is associated with irreversible damage to our bodies on cellular and organ levels and affect is essentially immediate. That means that if you eat even one unhealthy meal, damage to your body occurs right away on a cellular level.

There are many applications in the market today that provide medical, health and lifestyle suggestions and programs to prospective patients. Some of these applications currently on the market are typically provided by specialized medical and diagnostic clinics, screening diagnostic centers, and diet subscription services but are very disorganized and incomplete and carry significant price tags and thus affordable to mostly wealthy clients.

What is needed is a software application that provides one turnkey all-in-one complete and affordable solution which could be accessed by millions of consumers worldwide and will achieve this goal by utilizing simple lifestyle recommendations and behavior modification, existing cost-effective technologies including developed algorithms based on generative artificial intelligence (AI), telemedicine and direct access to cost-effective compounded medications as well as other treatments.

### SUMMARY

Disclosed herein is a personalized medicine application using, in some embodiments, blockchain, personalized telemedicine, generative Artificial Intelligence (“AI”), machine learning, population health and other technologies to help extend human life and longevity and reduce the user's rate of aging. The present disclosure analyzes multiple separate factor groups including (1) given fixed factors (i.e., “nature” such as family history and genetics); (2) current medical history (known and unknown or newly discovered via specialized testing); (3) non-fixed or external factors (i.e., “nurture”) in order to make recommendations for lifestyle modifications (for example, sleep, exercise, stress reduction, etc.) and other controllable factors, including dietary and nutritional changes; and (4) epigenetic enhancement techniques including simple techniques (such as adding certain supplements and medications) and more complex current and any future techniques (plasma transfusion, stem cells, etc.). The present disclosure allows patients (also referred to as “users” throughout this disclosure) to improve their diet, lose weight (if needed), prevent or control diseases, lower their biological age, extend their healthspans and reduce their rage of aging.

The term life span (or “lifespan”) refers to the total number of years a person lives, from birth to death (i.e., longevity). The term “healthspan” as used in the present disclosure is focuses on the number of those years that a person remains free of significant illness or disease. The term healthspan as used herein emphasizes a longer, healthier, better, more fulfilling life and not simply a longer life, as measured by actual years. Healthspan includes focusing on physical, cognitive, and emotional well-being to avoid prolonged decline and to maintain the person's vitality.

The present disclosure advantageously aggregates personal/patient data to create various unique AI-based health span/longevity algorithms and to discover important insights about the patient. Once these algorithms are working at scale, they can be offered to the user and facilitate service to pharmaceutical, insurance and other medical and non-medical companies.

The present disclosure also provides “predictive analytics” in the field of healthcare longevity/healthspan. In one embodiment, the calculated algorithms can predict how a user will look in the future, given a set of input data for that user. In other embodiments, calculated algorithms including, in some embodiments, AI and machine learning, can determine the user's rate of aging, and provide suggestions on how the user can modify their lifestyle to reduce this rate of aging. Predictive analytics uses historical data, statistical algorithms, and machine learning to forecast future outcomes and trends. It goes beyond descriptive analysis by using current and past data to predict what is likely to happen to the user, based on their input date.

The present disclosure can be incorporated into existing devices such as so-called “smart glasses” or “smart watches.” In other embodiments, users will be able to use the inventive methods disclosed herein and be able to, for example, scan restaurants or menus or food items either live (i.e., when driving or walking down the street looking for a restaurant) or shopping in the supermarket, food delivery services or from an online search to select the healthiest options for that user.

The present disclosure provides guidance to users and makes recommendations for certain diagnostic tests including medical imaging and provides routine monitoring via advanced testing including epigenetic analysis. Many users are not aware that they are afflicted with certain conditions until the later stages of their lives. Sometimes this is too late. Through specialized testing and/or screening, the present disclosure helps to uncover various medical conditions often during preclinical stages.

The present disclosure provides a flexible and stackable solution, and there is no specific cost requirement expected from the users. It is meant to be accessible to as many people as possible globally and is available as a free and very low-cost solution for the masses. Premium clients can spend as little as a few hundred dollars per month or less, depending on how much they are able to afford and their level of urgency to make important changes and specific interest or involvement. They can accelerate anytime and there is no requirement to spend any significant money at once unlike existing solutions on the market.

At the core of the system of the present disclosure is the concept of epigenetics, which is a link between “nature and nurture” and is a rapidly evolving science which states that a person's experiences (nature) can impact a person's life (nurture). Epigenetic biomarkers are sensitive to the environment and control how our DNA functions. They can be temporary or permanent and can affect how genes are switched on or off.

Epigenetics can help explain why identical twins may behave very differently or have different physiological responses, even though they have the same genotype. It can also help explain how environmental factors, like diet, smoking, pollution and stress, can lead to DNA-level changes that are linked to health disparities.

In accordance with one aspect of the present disclosure, a computer-based method for extending a user's healthspan and decreasing the user's rate of aging, is provided. The method includes receiving user information via a lifestyle platform, the user information including identification information, medical information, and at least one of biometric information, lab information, medical imaging reports and images of the user, processing and analyzing the received information using at least one of AI technology, machine learning and algorithms, providing accurate health, wellness and longevity information and recommendations to the user.

In some embodiments of this aspect, the computer-based method further includes using a medical data management system having blockchain technology to secure the information provided by the user. In some embodiments, the processing and analyzing step includes consideration of one or more of genetic and fixed factors, the user's current health status and external factors, including the user's diet and lifestyle. In some embodiments, a first healthspan score is determined based upon the genetic and fixed factors and the user's current health status, and wherein the first healthspan score can be increased depending upon the external factors to result in a final healthspan score.

In some embodiments, the final healthspan score is adjusted depending upon one or more of the sex of the user, at least one of the height and waist-adjusted body mass index (wBMI) of the user, the user's family health history, certain current medical conditions of the user, and a current physical activity level of the user. In some embodiments, the method further includes integrating the user information with hospital and medical facilities. In some embodiments, the method further includes determining the user's rate of aging based upon the received user information, the rate of aging being one of an average rate of aging, an accelerated rate of aging and a decelerated rate of aging.

In some embodiments, the method further includes estimating the user's future physical appearance. In some embodiments, the method further includes receiving, from the user, user-specific goals including at least one of losing a specific amount of weight, eliminating one or more medical conditions, and achieving a desired rate of aging within a specified period of time. In some embodiments, the method further includes providing an estimate of one or more of the user's physical characteristics and age progression based at least upon the images of the user. In some embodiments, the method further includes identifying the user's predisposition in developing certain medical conditions, and providing recommendations how to eliminate, avoid and/or limit the certain medical conditions. In some embodiments, the method further includes utilizing at least one of biometric devices, smart watches and smart glasses to present to the user the accurate health, wellness and longevity information and recommendations and rate of aging determination. In some embodiments, the received user information includes current diet, the method further includes providing to the user a recommended improved diet and using the recommended improved diet to calculate revised health, wellness and longevity information and to provide updated rate of aging information. In some embodiments, the method further includes providing one or more notifications to the user to remind the user to one or more of access the platform more frequently, maintain the health, wellness and longevity information and recommendations, and provide additional requested information.

In accordance with another aspect of the present disclosure, a computer-based system for extending a user's healthspan and decreasing the user's rate of aging, is provided. The system includes processing circuitry including one or more processors, memory and a communication interface. The communication interface is configured to receive user information including identification information, medical information, and at least one of biometric information, lab information, medical imaging reports and images of the user. The one or more processors are configured to process and analyze the received information using at least one of AI technology, machine learning and algorithms. The communication interface is configured to provide accurate health, wellness and longevity information and recommendations to the user.

In some embodiments of this aspect, the system further includes a medical data management system having blockchain technology to secure the information provided by the user. In some embodiments, the processing and analyzing comprises consideration of one or more of genetic and fixed factors, the user's current health status and external factors, including the user's diet and lifestyle. In some embodiments, a first healthspan score is determined based upon the genetic and fixed factors and the user's current health status, and the first healthspan score can be increased depending upon the external factors to result in a final healthspan score.

In some embodiments, the final healthspan score is adjusted depending upon one or more of the sex of the user, at least one of the height and waist-adjusted body mass index (wBMI) of the user, the user's family health history, certain current medical conditions of the user, and a current physical activity level of the user. In some embodiments, the one or more processors are further configured to determine the user's rate of aging based upon the received information, the rate of aging being one of an average rate of aging, an accelerated rate of aging and a decelerated rate of aging.

### BRIEF DESCRIPTION OF THE DRAWINGS

A more complete understanding of embodiments described herein, and the attendant advantages and features thereof, will be more readily understood by reference to the following detailed description when considered in conjunction with the accompanying drawings wherein:

FIG. 1 is a block diagram of an example system according to some embodiments of the present disclosure;

FIG. 2 is a block diagram of an example computing environment of FIG. 1 according to some embodiments of the present disclosure;

FIG. 3 is a flowchart of an example process performed by the computing environment according to one or more or more embodiments of the present disclosure; and

FIG. 4 is a flowchart showing the overall flow of the process of the present disclosure starting with patient intake information through database storage and AI analysis;

FIG. 5 continues from the flowchart of FIG. 4 showing exemplary tools available to the user;

FIG. 6 continues from the flowchart of FIG. 5 showing the output and display after AI analysis of the intake information; and

FIG. 7 is a flowchart of an exemplary process in accordance with one or more embodiments of the present disclosure.

### DETAILED DESCRIPTION

Before describing exemplary embodiments in detail, it is noted that the embodiments reside primarily in combinations of apparatus components and processing steps related to a method and system for recommending personal lifestyle and diet modifications based on medical and genetic inputs. Accordingly, the system and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

As used herein, relational terms, such as “first” and “second,” “top” and “bottom,” and the like, may be used solely to distinguish one entity or element from another entity or element without necessarily requiring or implying any physical or logical relationship or order between such entities or elements. The terminology used herein is for the purpose of describing particular embodiments only and is not intended to be limiting of the concepts described herein. As used herein, the singular forms “a”, “an” and “the” are intended to include the plural forms as well, unless the context clearly indicates otherwise. It will be further understood that the terms “comprises,” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

In embodiments described herein, the joining term, “in communication with” and the like, may be used to indicate electrical or data communication, which may be accomplished by physical contact, induction, electromagnetic radiation, radio signaling, infrared signaling or optical signaling, for example. One having ordinary skill in the art will appreciate that multiple components may interoperate and modifications and variations are possible of achieving the electrical and data communication.

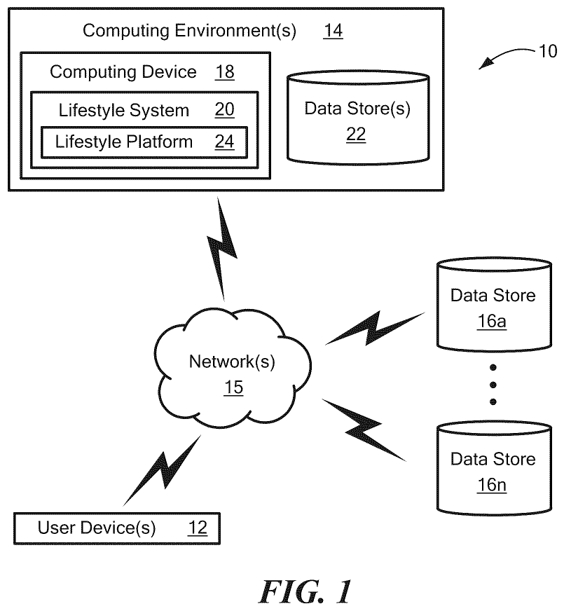

With reference to FIG. 1, shown is a block diagram of an exemplary system 10 according to some embodiments of the present disclosure. System 10 may include one or more user devices 12 and one or more computing environments 14 that may be in communication with each other via one or more networks 15 (collectively referred to as network 15). Further, system 10 may include one or more data stores 16a-16n (collectively referred to as data store 16) that are in communication with one or more elements of system 10, such as for example, the computing environment 14.

In one or more embodiments, user device 12 may refer to one or more of a computer, laptop, mobile phone, tablet, handheld electronic device, etc. User device 12 is configured to interface and/or communicate with computing environment 14 via network 15, as described herein. User device 12 may include one or more user interfaces (e.g., buttons, touch screen, input devices, etc.) to facilitate a user interacting with user device 12.

Further, computing environment 14 may include one or more computing devices 18. In embodiments using multiple computing devices 18, the computing devices 18 may be located in a single installation or may be distributed among many different geographic locations (e.g., cloud computing environment).

Further, computing environment 14 may include lifestyle recommendation system (also referred to as “lifestyle system”) 20 and data store(s) 22 for storing one or more models and/or input data, among other data that may be used by computing environment 14 to perform one or more functions described herein. Lifestyle recommendation system 20 is configured to provide lifestyle, health, wellness and longevity recommendation services based on one or more user inputs determined by lifestyle recommendation system 20, as described herein. In one or more embodiments, lifestyle recommendation platform (also referred to as “lifestyle platform,” “application,” or “platform/application”) 24 is part of and/or a sub-component of lifestyle recommendation system 20. In one or more embodiments, lifestyle recommendation system 20 is configured to process the various input data received and to and provide at least one output that indicates lifestyle and health goals, progress, and recommendations for a particular user (i.e., “patient”), as described herein. Further, lifestyle recommendation system 20 may be configured to perform other functionality related to determining input data such as by querying and/or searching one or more data stores 16.

As discussed in greater detail below, in one embodiment, lifestyle recommendation system 20 and lifestyle recommendation platform 24 use Artificial Intelligence (“AI”) to analyze some or all of the data received from the patient to provide a wealth of lifestyle, wellness, and health information and recommendations to the user.

Continuing to refer to FIG. 1, data store 22 of computing environment 14 may be configured to store various information and/or data associated with providing lifestyle and health goals and recommendations, as described herein. For example, data store 22 may store at least one lifestyle and health criterion (e.g., rules, thresholds, weights, etc.), model(s), input data, previous outputs/results generated by lifestyle recommendation system 20, among other data that may be used by lifestyle recommendation system 20 for performing the analysis described herein.

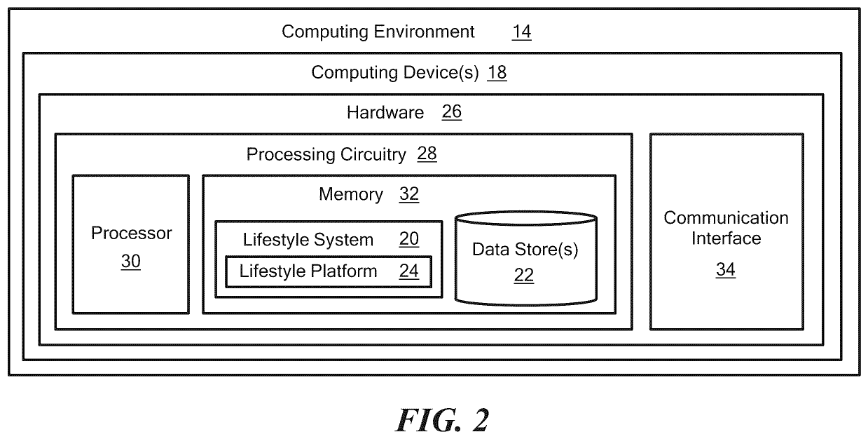

FIG. 2 is a block diagram illustrating the example computing environment (or “computer system”) 14 according to various embodiments. As shown, the computing environment 14 may include one or more computing devices 18. As shown, each computing device 18 comprises hardware 26. The hardware 26 may include processing circuitry 28. The processing circuitry 28 may include one or more processors 30 and one or more memories 32. Each processor 30 may include and/or be associated with one or more central processing units, data buses, buffers, and interfaces to facilitate operation. In addition to or instead of a processor 30 and memory 32, the processing circuitry 28 may comprise other types of integrated circuitry that perform various functionality. Integrated circuitry may include one or more processors 30, processor cores, FPGAs, ASICs, GPUs, SoCs, or other components configured to execute instructions. The processor 30 may be configured to access (e.g., write to and/or read from) the memory 32, which may comprise any kind of volatile and/or nonvolatile memory, e.g., cache, buffer memory, RAM, ROM, optical memory, and/or EPROM. Further, memory 32 may be embodied in the form of one or more storage devices. The processing circuitry 28 may be configured to perform various functionality described herein. For example, computer instructions may be stored in memory 32 and/or another computer-readable medium that, when executed by processor 30, causes the processor 30 to perform various functionality.

Hardware 26 may include communication interface 34 facilitating communication between one or more elements in system 10. For example, communication interface 34 may be configured for establishing and maintaining at least a wireless or wired connection with one or more elements of system 10 such as network 15, data store 16, user device 12, etc.

The processing circuitry 28 may be configured to control any of the methods and/or processes described herein and/or to cause such methods, and/or processes to be performed, e.g., in computing environment 14. Processor 30 corresponds to one or more processors 30 for performing computing device 18 functions described herein.

The memory 32 is configured to store data, such as files, data, information, etc. that are described herein. Also stored in the memory 32 and executable by the processor 30 are the lifestyle recommendation system 20 and lifestyle recommendation platform 24. Although FIG. 2 shows the lifestyle recommendation system 20 and lifestyle recommendation platform 24 being in a single computing device 18, the lifestyle recommendation system 20 and lifestyle recommendation platform 24 may execute in multiple computing devices 18 of the computing environment 14. To perform the functionality of the lifestyle recommendation system 20 and lifestyle recommendation platform 24, the memory 32 may include instructions that, when executed by the processor 30 and/or processing circuitry 28, causes the computing device 18 to perform the functionality performed by the lifestyle recommendation system 20 and lifestyle recommendation platform 24 described herein.

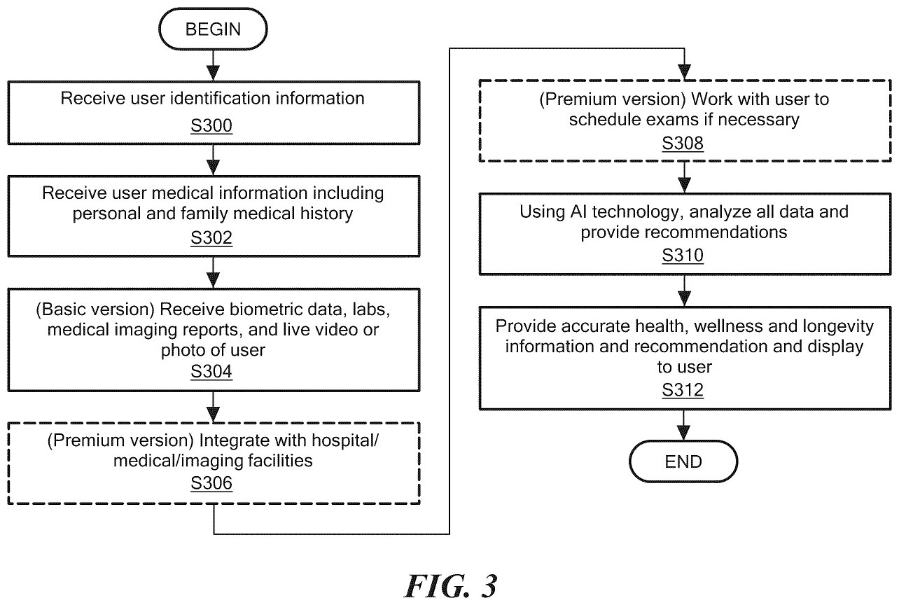

FIG. 3 is a high-level flowchart of an exemplary process performed by the computing environment according to one or more or more embodiments of the present disclosure. In one embodiment, lifestyle recommendation platform 24 receives user identification information, such as, for example, the user's name, gender, data of birth, address, contact information, etc., at step S300. Lifestyle recommendation platform 24 then receives, via a user interface on the user's computing device 12, the user's basic medical information, at step S302. In one embodiment, the user is presented with a questionnaire on their computing device 12. The information received from the patient may include personal and family medical history as well as basic information regarding the patient's overall health, fitness, diet and medical data including age, sex, food allergies, food intolerances, height/weight/waist-adjusted body mass index (wBMI), etc.

In one embodiment, if the patient has subscribed to the basic version of the application, in addition to the information above, lifestyle recommendation platform 24 will also collect biometric data, labs, and medical imaging reports, etc., at step S304 In one embodiment, video or photos of the patient will also be collected for analysis, since many conditions can be determined by analyzing the patient's appearance, i.e., facial expression, eyes. skin, body weight, gait, etc.

In one embodiment, if the patient has instead subscribed to the premium version of the application, lifestyle recommendation system 20 will integrate the information provided in step S304 with hospital and medical facilities, including Electronic Medical Records (“EMRs”) as well as imaging facilities to obtain Picture Archiving and Communication/Radiology Information Systems (“PACS/RIS”), Radiology, Pathology, Ophthalmology and other AI systems, at step S306. Additionally, lifestyle recommendation platform 24 will request and obtain from these facilities the actual medical images (for interpretation and second opinions), cellular analysis, genetic/DNA analysis, laboratory analysis, epigenetic analysis, microbiome analysis, pathology analysis and any additional advanced data which may be needed. lifestyle recommendation platform 24 will assist premium member patients in scheduling specific exams if they are not yet available, such as Computed Tomography (CT) Chest and/or CT Abdomen, full body MRI, labs, including cancer screening tests, genetic and microbiome analysis, etc., as well as epigenetic testing, at step S308. Most of these tests will be conducted with the patient's convenience in mind (e.g., test kits shipped to a patient's home). All of the testing results are conveniently available online.

Using AI technology, longevity recommendation system 20 processes and analyzes all of the data retrieved from the user and provides recommendations to the user, at step S310. The recommendations include providing accurate health, wellness and longevity information to the user and also to provide progress reports. This information is displayed to the user, at step S312, on the user's computing device 12.

The processing and analysis process of step S310 and the output process of step S112 will now be discussed in greater detail. All of the steps discussed below can be performed by computing environment 14, and specifically, hardware 26, including processing circuitry 28, processor 30, memory 32, lifestyle recommendation system 20 and lifestyle recommendation platform 24, data store 22 and communication interface 34.

### Process and Output

Lifestyle recommendation system 20, using software and generative artificial intelligence technology analyzes all available data within each category and provide certain recommendations with regards to diet and longevity. For example:
Video/photos of the patient;
Eye/retinal imaging of the patient;
Biometric data (from, for example, personal electronic health and fitness tracking devices) from sensors including but not limited to sleep and exercise patterns;
Radiology/diagnostic imaging reports or images;
Genetic reports;
Laboratory data including certain cancer screening tests;
Pathology reports;
Microbiome analysis;
Epigenetic cellular reports (telomere length, DNA methylation);
Any other advanced health and medical data

### Patient Appearance

Software run on processor 30 is configured to use AI to analyze patients'appearance from videos/photos to determine if patients suffer from any medical conditions and disease such as but not limited to hypertension (high blood pressure), thyroid disease/masses, stroke, obesity, The general appearance of a patient may provide diagnostic clues to the illness, severity of disease, and the patient's values, social status, and personality. There are many overt signs of illness: changes in turgor [elasticity] or consistency of the skin that can show maladies of the heart; changes in skin color that can be indicative of liver disease; and lips, mouth, and tongue can be indicative of nutrition deficiencies. AI can also determine the relationship to chronological age, apparent state of health and apparent state of comfort and mental status.

### Patient's Eyes

By examining a patient's eyes/retina, lifestyle recommendation system 20 uses AI to detect conditions such as diabetes, high blood pressure, arterial plaque, multiple sclerosis, brain tumors, stroke, leukemia and many other conditions.

### Radiology/Diagnostic Imaging

Likewise, lifestyle recommendation system 20 uses AI-based software to analyze medical images and/or reports for various conditions and diseases (for example, fatty liver, atherosclerosis, coronary arterial disease, atherosclerosis osteoporosis, chronic lung conditions, fractures, etc.) and determines the patient's biological age (as opposed to chronological age) based on available research data.

### DNA Testing

Lifestyle recommendation system 20 analyzes data to determine the presence of any genetic conditions such as, for example, cystic fibrosis, Tay-Sachs disease, sickle cell disease, Down syndrome, spina bifida, Turner syndrome, etc. Genetic testing can also predict the risk of developing certain conditions, such as cancer. Genetic testing can also help diagnose neurological diseases, such as Alzheimer's disease, Parkinson's disease, and amyotrophic lateral sclerosis (ALS).

### Microbiome Testing

A microbiome test, also known as a “gut health” test, measures the microorganisms in a person's stool to identify the types and levels of bacteria, viruses, and fungi in their stomach. The test involves collecting a stool sample, which is then sent to a lab for analysis. Microbiome testing measures the different levels of microorganisms present in the GI tract. More than 200 types of bacteria, viruses, and fungi are present in the human GI tract. Research suggests that these microorganisms may play a role in chronic conditions such as: inflammatory bowel disease (IBD). Similar examples can be shown within other areas including biometric data and labs.

### Epigenetic Cellular Analysis—Telomere, DNA Methylation, Protein Analysis etc.

Information from certain available cellular tests (telomere length, DNA methylation etc.) can be analyzed. Aberrant DNA methylation has been implicated in many disease processes, including cancer, obesity, and addiction. DNA methylation tests can identify single nucleotide polymorphisms (SNPs), which are genetic variations that influence a patient's health and risk of chronic illness. Abnormal methylation patterns may indicate an increased risk for conditions like cardiovascular disease, neurodegenerative disorders, or hormonal imbalances.

Telomere testing measures the length of telomeres in a person's blood sample to provide information about their biological age and overall health. Telomeres are protective caps at the tips of chromosomes that shorten with each cell division. Shortened telomeres can lead to impaired tissue replenishment and cell death. Telomere testing can help people take steps to preserve cellular health and prevent disease. It can also help assess the risk of age-related diseases, such as premature coronary heart disease.

### Generative AI Process

All of the available data received by lifestyle recommendation system 20 can be combined to provide an accurate person's health, wellness and longevity calculation. The generative AI algorithm (described below) makes certain recommendations to the patient and puts them into one or more treatment pathways:
Healthy diet and weight loss
Longevity extension
Prevention of specific chronic diseases to which the person is at risk or predisposed to

### Healthy Diet and Weight Loss Pathway

Free version. In one embodiment, general recommendations and personalized health information are provided for specific types of diet, for example, Gluten Free, Vegan, Keto, Gout friendly, high blood pressure, diabetes etc. In one embodiment, there is the ability to upload pictures of food item packaging or to scan a barcode of all food items and general recommendations regarding specific food items. There will be targeted recommendations for specific food choices in specific local restaurants and supermarkets in the area. In one embodiment, recommendations for healthy recipes will be provided.

Paid Basic Version. One embodiment will provide the ability to scan barcodes of food items, and an application will rate the item based on personalized data, not simply generic recommendation. There will be targeted recommendations for specific food choices in the specific local restaurants and supermarkets in the area. Recommendations for healthy recipes based on personalized dietary recommendations will be provided.

Premium Version. The Premium Version provides the features discussed above plus telemedicine access to medical doctors who will be able to prescribe certain medications (e.g., Semaglutide and other Glucagon-Like Peptide-1 (GLP1) medications) as well as supplements to assist with weight loss. Partner pharmacies will mail highly personalized/customized compounded medications and supplements monthly.

### Longevity Pathway

Free and Paid Basic Versions. Longevity will be calculated using proprietary algorithms based on the input data, available research and existing actuarial tables. Every modification in diet, exercise and other external factors will adjust the longevity calculator using research data. Premium members will have the above feature (in #1) plus a prediction tool to extend lifespan based on various scenarios. One embodiment provides access to a network of specialized clinics including physicians, dieticians and other medical professionals based on personalized health information. These clinics and medical professionals will be available onsite and via telemedicine, depending on specific recommendations and needs. Clinicians will recommend and prescribe certain life extending medications (e.g., Sodium-Glucose Cotransporter 2 (SGLT2), GLP1, Metformin, Rapamycin and other medications) and provide treatments as needed (stem cells, etc.). Partner pharmacies will mail highly personalized/customized compounded medications and supplements monthly.

### Disease Prevention and Control Pathway

In one embodiment, this feature will only be available for premium members. Generative AI software will identify any chronic diseases or conditions that a person suffers from (and may or may not be aware of), predisposed to, or at risk of developing. Certain medications will be prescribed by physicians via telemedicine. For example, lifestyle recommendation system 20 determines that a person has osteoporosis after examining CT (using, for example, AI algorithms). Therefore, it is determined that the patient will benefit by taking bisphosphonate medications (Fosamax, etc.) Partner pharmacies will mail highly personalized/customized compounded medications and supplements monthly.

### Backbone Blockchain Technology

In one embodiment disclosed herein, is a medical data management system using secure blockchain technology allowing users to retain ownership over their own records while allowing clinics and other 3rd parties to have easy access to it. Blockchain protocols will be utilized to exchange and collect medical data. Blockchain is a decentralized ledger used in healthcare to store and share patient data securely and efficiently. It can help improve the quality of care by making it easier to manage patient data across fragmented healthcare systems.

A blockchain network is used in the healthcare system to preserve and exchange patient data through hospitals, diagnostic laboratories, pharmacies, and physicians. Blockchain applications can also accurately identify severe mistakes and even dangerous ones in the medical field.

The method combines Internet of Medical Things (“IoMT”) and blockchain to encrypt and save the user's health information. Online questionnaires, and technologies such as AI, smart sensors, etc. collect the user's health data, and then encrypted health data will be stored in the nodes of the Ethereum blockchain, thus protecting the privacy of users.

### Tokens

In one embodiment, the system disclosed herein utilizes crypto tokens which can enhance efficiency and trust within a system by facilitating transactions and granting access. They can also democratize access to assets and provide online transparency and security. Each paid member will receive a certain number of tokens which they can exchange for specific services. Also, if they follow a healthy lifestyle recommended by the app, not only will it improve their longevity, but will also receive bonus tokens which they can further exchange for additional life extending services, therefore this becomes a positive reinforcement cycle.

Blockchain has several characteristics that make it appealing for healthcare, including:
Decentralization: No central authority controls the content added to the blockchain.
Smart contracts: Self-executing contracts that don't require a central authority.
Audit and traceability: Blockchain can link new blocks to previous blocks, forming a chain of blocks.

Advantages to using blockchain:
Secure data sharing. Blockchain can enable secure data sharing between multiple parties, including healthcare providers and patients.
Improved interoperability. Blockchain can help improve interoperability between healthcare systems.
Better patient care. Blockchain-based medical records can create an ecosystem of patient data that can be used to improve diagnoses and personalized care plans.
Supply chain organization. Blockchain can be used to organize supply chains and prevent counterfeit prescriptions and devices.
Improved electronic health records. Blockchain can connect electronic health records (EHRs) and distribute property of the records across all stakeholders.

Software utilized by lifestyle recommendation system 20 includes algorithms utilized to perform the analysis in step S310 and provide the output in step S312 of FIG. 3. Some examples of these algorithms are discussed below.

### Algorithms—Healthspan Calculation

In one embodiment, the algorithms disclosed herein are an interplay between separate factors. For example, these may include:
1. Genetic/fixed factors-male/female, age, family history, etc. Weighting 25% of total maximum points.
2. Current patient's health (medical conditions, etc.). Weighting 25% of total maximum points.
3. External factors-diet, lifestyle, etc. Weighting 25% of total maximum points.
Points/years are vested every quarter based on compliance with these factors.

Epigenetic monitoring and enhancement. Weighting 25% of total maximum points.

After analyzing all factors, the algorithm will compute the expanded healthspan, the maximum number of points that each individual can earn (goal or runway) as well as actual points earned with every change in lifestyle. Not including bonus points (TBD below), the ultimate goal is earning up to, for example, 2,000 points (not including bonus) with 100 points equaling one additional year of longevity added. It should be noted that the point totals and scenarios disclosed herein are exemplary only and are used only to illustrate the various algorithms.

The first 2 factors (fixed and current conditions) determine the maximum achievable goal. External factors determine how many actual points a patient earns every quarter. If all factors are satisfied then the maximum number points are added. Satisfying all external factors essentially implies that all medical conditions are well controlled and external factors can counteract the first two factors. Additionally, the maximum achievable goal can be expanded in some cases when certain treatments or medications that expand longevity, bonus points. This process can be monitored with epigenetic testing, which is discussed below.

As an example, a 30-year old female with an achievable goal of 2,250 points, can in fact expand her achievable goal up to 3,000 points if external factors are fully satisfied and bonus points are added.

In one embodiment, there is a circular progress bar that can be used as a snapshot of the application. For example, if 1,000 points are achieved out of total 2,000 goal, new points will be added every three months if all the goals are met. In one embodiment, using different colors can indicate if there is success, danger (if they are not achieving their goals) or warning etc.

In another embodiment, if a user fails to achieve goals for a certain number of consecutive periods of time, (e.g., two straight quarters), lifestyle recommendation system 20 adjusts the user's final number of achievable points accordingly. For example, if a particular user has a 2,000 maximum achievable points and they fail to meet their goals, they may lose 200 points, which lowers their total achievable points to 1,800. The user can potentially catch up and recover that and even earn bonus points.

### Fixed Factors

Genetics, family history and fixed factors. This refers to an inherited genetic variation that increases an individual's likelihood of developing a specific disease or health condition, indicated by fixed factors (such as sex, height, wBMI, weight, etc.), genetics as well as family history, where genes passed down from parents can contribute to susceptibility to certain illnesses like cancer, heart disease, diabetes, or autoimmune disorders. Essentially, it's the probability of getting a disease based on the patient's genetic makeup. Some major factors are discussed herein. However, this discussion is not considered all inclusive.

### Age

In one embodiment, for example, healthy females will have a goal of earning up to 2,250 points depending on their current age and sex. For example, 100 points =1 year added. If the patient starts maintaining healthy living (external factors discussed above) on or by age 40, a full 2,250 points could be added as a goal. If the patient starts maintaining a very healthy lifestyle by age 50, 1,700 points could be added as a goal. These are examples only and may be prorated using AI based software.

### Sex

In one embodiment, males will have an adjustment of 0.88.

### Height/BMI

Taller and larger individuals generally have shorter lifespans compared with their shorter and smaller peers. This is in part due to the fact the shorter and smaller peers have less cells and less things that could go wrong so to speak. In individuals who lived for at least 50 years, longevity is inversely correlated with adult height in men. Some studies have found that shorter and smaller people are less likely to die from certain diseases, like cancer, and live longer lives. For example, a study of professional baseball players found that shorter men had a 10-15% longer life span.

### Family History

Family history tends to be a strong indicator of genetic risk, as close relatives sharing similar genes can increase the likelihood of inheriting a predisposition to a disease. As an example, a family history of high blood pressure can increase one's risk of developing the condition and may shorten their lifespan. Having a close family member with high blood pressure before age 60 doubles your risk of developing it. If three or more relatives had high blood pressure before age 60, that represents a strong family history of high blood pressure. A Mendelian randomization (MR) analysis found that a 10 mm Hg increase in genetically proxied diastolic blood pressure (DBP) reduced lifespan by 3.09 years. This is a goal adjustment factor of, for example, 0.91.

### Gene Mutations

Specific changes in DNA sequences that can directly contribute to disease development. Examples of genetic risk factors:
Familial hypercholesterolemia (FH): A genetic condition leading to high cholesterol levels, increasing the risk of heart disease. People with FH have a higher risk of heart disease and death at a younger age. Without treatment, life expectancy can be reduced by 15-30 years. In the case of homozygous FH, a rarer and more severe type of the condition, life expectancy may be only 20 years or less. Ratio adjustment of 0.5.
BRCA gene mutations: Associated with increased risk of breast and ovarian cancer. A study using mortality data from relatives of participants in the Washington Ashkenazi Study found that female carriers of BRCA1/2 mutations have a life expectancy that is 6.8 years lower than non-carriers. Ratio adjustment of 0.7.

### Current Health/Medical Conditions

Known medical conditions are collected from history (known) as well as any new conditions discovered via medical and screening testing as described above. Patient's current health together with fixed factors will dictate the goal (or total achievable points). As stated above, in one exemplary embodiment, healthy females will have a goal of earning up to 2,250 points depending on their current age and sex. 100 points =1 year added, while males have an adjustment of 0.88. Each earned point will result in tokens which can be redeemed for purchases in the network (receive discounts for testing, supplements, etc.). Various medical conditions and prior medical events will have an adjustment ratio between 0.999 to 0.1 from minimal to severe. Conditions are additive (multiple ratios). For example:
Minimal 0.971-0.999
Mild 0.83-0.97
Moderate 0.71-0.82
Severe below 0.7

### Example of Conditions:

Celiac disease 0.997
Blood pressure (not controlled) 0.97
Moderate obesity 0.91
Diabetes 0.91
Osteoporosis 0.88
Stroke 0.85
Heart attack 0.73
5 mild to moderate chronic conditions 0.76
Severe obesity 0.7

### Example of Adjustments

A 60-year-old male with diabetes and osteoporosis will have an adjustment to maximum achievable points as follows: 2,250×0.88 (male)×0.91 (diabetes)×0.88 (osteoporosis)=1400 (rounded) points.

External factors: Total weight up to 1.0

According to current research, the most important external factors for longevity include: a healthy diet, regular exercise, maintaining a healthy weight, not smoking, managing stress, strong social connections, and access to quality healthcare; essentially, adopting a healthy lifestyle with positive behavioral choices is key to increasing lifespan. In one embodiment, external factors can be grouped into 6 main categories. Each external factor has a different weight, totaling to a maximum of 1.0.

Diet: weight 0.3

One of the most important and complex categories is diet. As they say: “You are what you eat.” Eating a balanced diet rich in fruits, vegetables, and whole grains can significantly contribute to longevity. Maintaining a healthy eating pattern means getting the daily recommended amounts of vegetables, fruit, nuts, whole grains, polyunsaturated fatty acids, and omega-3 fatty acids and limiting red and processed meats, beverages with added sugar, trans fat, and sodium. Studies have found that the Mediterranean Diet is associated with a longer life expectancy and a reduced risk of death from various causes.

One study found that adopting the Mediterranean Diet alone at age 20 could increase life expectancy by 13 years for men and 10.7 years for women. Another study found that people who adhered more closely to the Mediterranean Diet had a longer lifespan, with those who adhered less having a lifespan that was 5-8 years shorter. A study of 25,315 women found that those who closely followed the Mediterranean diet had a 23% lower risk of early death. The study also found that the risk of dying from cancer was reduced by 17% and the risk of dying from cardiovascular disease was reduced by 20%. The study also found evidence of biological changes that may explain the diet's health benefits, including changes in biomarkers of metabolism, inflammation, and insulin resistance.

A good diet is so critical, patients with an exceptionally healthy diet will be able to earn bonus points (see discussion below). The reason for this is that diet alone can help manage and reverse many chronic conditions, including:
Celiac disease: Removing gluten from your diet can cure celiac disease in most cases.
Coronary artery disease: Plant-based diets can reverse coronary artery disease.
Type 2 diabetes: Very low carb lifestyles may help eliminate type 2 diabetes in some people.
Diet can also help prevent and manage other chronic conditions, including, for example: heart disease, cancer, obesity, diabetes, high blood pressure, osteoporosis, and Alzheimer's.
Smoking, drugs, alcohol habits and any external toxins—weight 0.15.
Smoking cessation or not smoking is considered one of the most impactful singular lifestyle changes for longevity. In addition to not smoking, drinking only moderate amounts of alcohol (one drink or less per day for women or two drinks or less per day for men, wine recommended) as well as not using drugs are crucial factors.
Physical activity and healthy weight—weight 0.15.

Physical activity includes both cardiovascular health as well as strengthening exercises. Getting at least 3.5 hours of moderate to vigorous physical activity each week is critical. The goal is to maintain a normal weight (body mass index between 18.5 and 24.9). Negative weight adjustments are on both spectrum-being obese as well as being too thin. It is interesting to note that being slightly overweight is not associated with longevity risks.

### Sleep—Weight 0.1

The recommended amount of sleep depends on your age, but generally, adults should get 7-9 hours of sleep per night, though some may need as few as 6 hours or as many as 10 hours. Getting enough sleep is important for your health and well-being. Not getting enough sleep can lead to health issues like weight gain, high blood pressure, and depression. People who sleep less than 7 hours or more than 9 hours may have 1-3 fewer years of good health and disease-free life

Stress management—weight 0.1

Effective stress coping mechanisms are crucial for overall health and longevity.

Social connection—weight 0.1

Strong social networks and meaningful relationships can positively impact lifespan. Married vs single, support network, friends, etc.

Single vs married—adjustment 0.85
Access to healthcare—weight 0.1

Regular checkups and timely treatment of health concerns can improve life expectancy. In one study, adequate access to healthcare increased life expectancy by about 2.0-2.5 years at age 65 and 1.0-1.2 years at age 85.

One research team collected data on all factors listed above and compared health outcomes for those who adopted all of the above with those who didn't adopt any. The researchers also collected information about the participants'medical history, such as heart disease, cancer, and diabetes, as well as when they died. At age 50, women who didn't adopt any of the healthy habits were estimated to live on average until they were 79 years old and men until they were 75.5 years. In contrast, women who adopted all healthy lifestyle habits lived 93.1 years and men lived 87.6 years.

Independently, each of these healthy lifestyle factors significantly lowered the risk of total death, death from cancer, and death from heart disease.

This study underscores the importance of following healthy lifestyle habits for improving longevity in the U.S. population. However, adherence to healthy lifestyle habits is very low. Adherence to a healthy lifestyle habits is one of the goals of the present disclosure.

In one embodiment, external factor data is continuously collected, and patients are awarded points on the quarterly basis. For example, a healthy female aged 40, with an achievable goal of 2,000 points will be awarded 250 points every quarter (1,000 per year) if she maintains a 100% healthy lifestyle. However, if she continues to smoke then she will only be awarded 200 points (0.8×250). It is important to realize that lifestyle modification is a long-term commitment and not a simple one-time cure or pill.

### Epigenetics, Epigenetic Testing and Bonus Points

Epigenetics is the study of how environmental and behavioral factors can impact how genes function without altering the DNA sequence. Epigenetic changes are chemical modifications that are added to DNA, regulating whether genes are turned on or off. These changes can be passed down through generations and can vary between individuals, tissues, and cells. Some examples of epigenetic changes include:
Diet. The Mediterranean Diet (and the absence of cigarette smoking) are correlated with longer telomere length.
Smoking. An epigenetic change associated with smoking can be reversed after a person quits.
Exercise. Research suggests that exercise may have positive effects on well-being by altering the epigenome.

Epigenetic tests can provide information on whether a person's gene expression has changed due to lifestyle habits or environmental changes. Epigenetic regulation is a reversible process that can determine or contribute to human diseases.

There are several types of epigenetic tests already available, and others are in development, including tests that measure telomere attrition and DNA methylation levels.

### Telomere Attrition or Lengthening

Telomeres are the protective caps at the end of each chromosome and play a vital role in maintaining genomic stability. Telomere length varies widely between individuals and depends on many factors, including age and health conditions.

Telomere results are reported as the average telomere length, which is the average number of base pairs in a telomere from all telomeres detected in a sample. The results are then compared to reference ranges based on age to interpret the results.

While telomeres naturally shorten over time with cell division, research suggests that lifestyle changes like regular exercise, a healthy diet, stress management, and social support may potentially lead to increase in telomere length, indicating a potential to slow down the rate of telomere shortening

Telomeres shorten with each cell division, and when they reach a certain length, the cell stops dividing or dies. Telomere attrition is linked to a wide range of age-related diseases.

Typical strategies for extending telomeres include: eating foods like legumes, nuts, seaweed, fruits, dairy products, and coffee and avoiding most alcohol, red meat, and processed meat.

The frequency of telomere testing depends on several factors, including your risk factors, lifestyle changes, and medical treatments. Some researchers recommend getting a telomere test at least once a year to compare results and track how your telomeres change over time. If you've made changes to your lifestyle or undergo certain treatments, you may be able to see changes in your telomeres in as little as six months. A telomere test is a blood draw or cheek swab that sends a sample to a lab for evaluation. The results can take a few weeks to come back.

### DNA Methylation Testing

DNA methylation analysis allows scientists to gain valuable insight into gene regulation and identify potential biomarkers. Aberrant DNA methylation has been implicated in many disease processes, including cancer, obesity, and addiction.

A DNA methylation test can provide information about how your methylation pathway is functioning and may help identify genetic variations that affect your health. DNA methylation is a chemical modification of DNA that can alter gene activity without changing the DNA sequence. DNA methylation analysis can help identify potential biomarkers for diseases like cancer, obesity, and addiction. DNA methylation can be a record of exposure to external factors like unhealthy food, smoking, and pollutants, as well as internal factors like stress and hormones. A DNA methylation test can also help identify if you have a methylation defect, and what nutritional support you may need to improve your methylation pathway.

### Longevity Treatments, Monitoring and Bonus Points

In one embodiment, premium members will have access and recommendations to new treatments (supported by years of research). These recommendations and treatments include:

### Senolytic Medications and Supplements

Supplements. Various supplements will be shipped such as creatine, various anti-oxidants such as polyphenols particularly a compound called hydroxytyrosol (active in specific types of olive oils), resveratrol (active ingredient of specific types of red wine), Ginkgo, Quercetin, ginger extracts, etc.

### Over the Counter Medications Like Aspirin

Medications will be prescribed via telemedicine according to specific patient needs and shipped to the door: (For example, Metformin, Rapamycin, GLP 1 analogs (Semaglutide, etc.), Sildenafil, Angiotensin II Receptor Blockers (ARBs) (Olmasartan/Losarten, etc.), SGLT2 inhibitors (empagliflozin), alendronate, etc.

### Life Extending Therapies (Optional)

Some of these therapies are already being performed in the US or outside (Bahamas, etc.). For certain patients, the disclosed system will work with certain reputable functional medicine and longevity clinics. Some of the procedures may include, for example:
IV therapy

### Cryotherapy

Stem Cell Therapies (many countries outside of USA)

### Cell Therapies

Telomere extension therapies, treatments that aim to extend the length of telomeres, the protective caps on the ends of chromosomes, performed as follows:
Hyperbaric oxygen therapy
Telomere lengthening procedure—Stanford University procedure that uses modified RNA to introduce the telomerase enzyme, which rebuilds telomeres. This procedure can increase telomere length by up to 1,000 nucleotides and may reverse the effects of aging in cells.
Telomerase gene therapy—therapy that involves injecting patients with viruses that carry the genetic instructions for producing telomerase.

### Transfusions.

The disclosed system will monitor beneficial effects of the supplements, medications and therapies (in addition to compliance with beneficial external lifestyle factors discussed above). The system will make recommendations for routine and appropriate follow up for epigenetic testing (in addition to other testing such as Full body MRI, microbiome or DNA testing, etc.).

Epigenetic changes can be flexible and rapid and can also be temporary or permanent. Epigenetic change triggered by environmental conditions may be reversed when those conditions change again. As discussed above, lifestyle factors such as diet, behavior, stress, physical activity, working habits, smoking, and alcohol consumption can influence epigenetic mechanisms.

In one embodiment, patients who demonstrate epigenetic improvements will earn up to 750 bonus points (i.e., a 7.5-year life extension). In some embodiments, the algorithms will provide demonstrable changes on diagnostic testing (like full body MRI), DNA analysis and epigenetics testing. Some ways to prove epigenetic improvement include:
Telomere testing: There is a strong scientific association between telomere length and longevity. Individuals who are able to reach 100% of telomere lengthening for their given age will be able to, for example, add up to 1,000 points.
Chromatin immunoprecipitation (ChIP): A technique that combines sequencing technology with DNA that has been immunoprecipitated with antibodies against specific histone modifications.
DNA methylation analysis: A technique that uses sequencing and array-based techniques to investigate methylation patterns across the genome.
DNA-protein interaction analysis: A technique that provides insight into DNA-protein interactions.
Chromatin accessibility analysis: A technique that uses ATAC-Seq to evaluate regions of open chromatin across the genome.

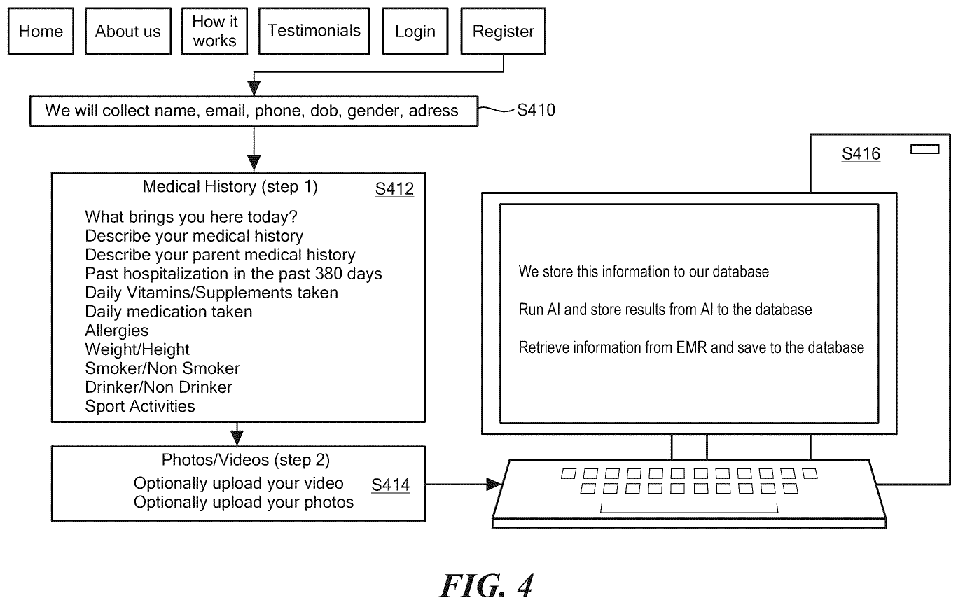

FIG. 4 is a flowchart showing the overall flow of the process of the present disclosure starting with patient intake information through database storage and AI analysis. In one embodiment, the patient is presented, via lifestyle recommendation platform 24 with a log on screen on their user device 12. The patient's contact and personal information is collected including, for example, their name email address, phone number, date of birth, gender, address, etc., at step S410. The user is then presented with a screen asking for additional information such as the patient's medical history, vitamin and/or supplement intake, whether they have allergies, or smoke, etc., at step S412. The patient is then presented with the option of uploading their photo at step S414. Finally, lifestyle recommendation system 20 stores the inputted patient information, retrieves additional information such as EMR and runs AI and/or the algorithms discussed herein at step S416.

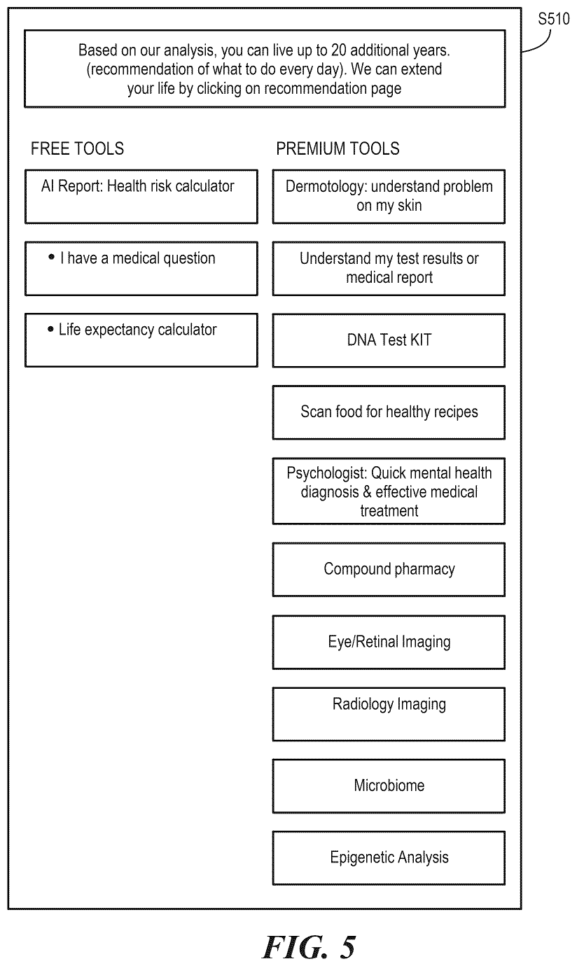

FIG. 5 continues from the flowchart of FIG. 4 showing exemplary tools available to the user. As described herein, depending upon the plan the patient has chosen, the patient is presented with options/tools to choose from, at step S510. As shown in FIG. 5, the free tools available to the patient are more limited then the tools available under the premium plan.

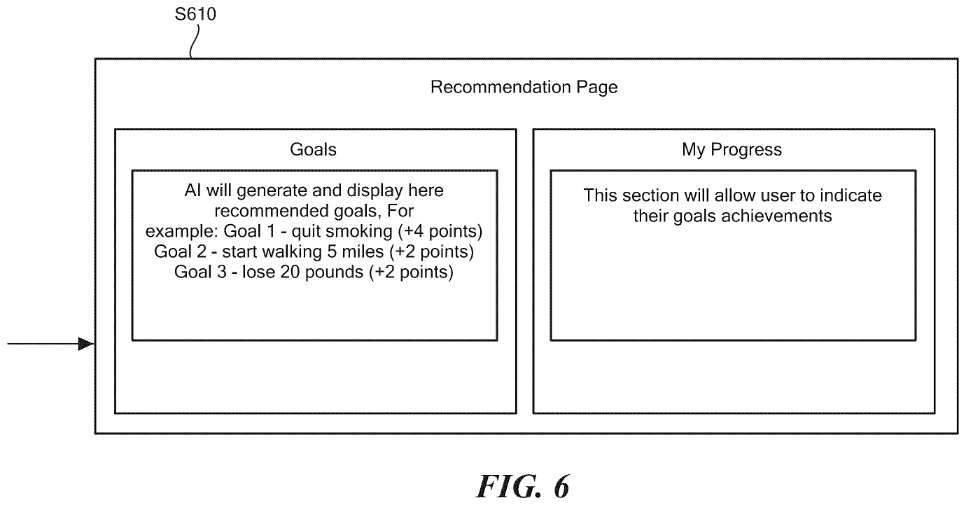

FIG. 6 continues the flowchart of FIG. 5 showing the output and display after AI analysis of the intake information. As discussed herein, lifestyle recommendation system 20 analyzes and processes the information obtained from the patient or from outside sources. Based upon this analysis, the patient is presented with recommendations, at step S610. These recommendations could include, for example, a listing of goals for the specific patient, as well as a section where the user can indicate their progress with respect to these goals.

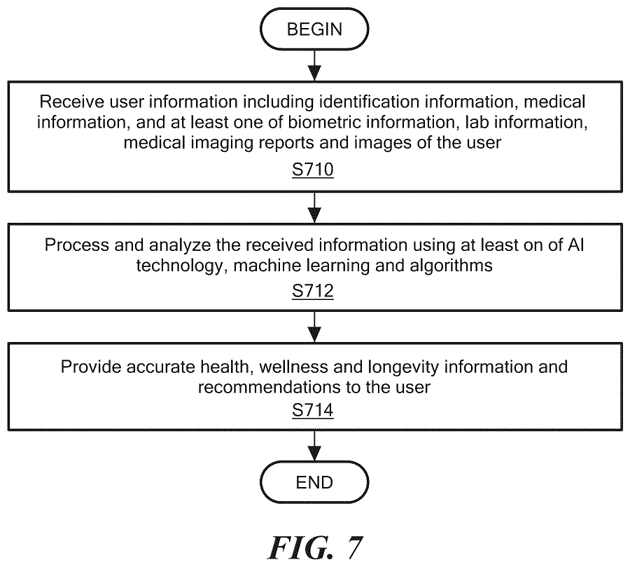

FIG. 7 is a flowchart of an exemplary process in accordance with one or more embodiments of the present disclosure. The process includes, receiving user information including identification information, medical information, and at least one of biometric information, lab information, medical imaging reports and images of the user, at step S710; processing and analyzing the received information using at least one of AI technology, machine learning and algorithms, at step S712; and providing accurate health, wellness and longevity information and recommendations to the user, ats step S714.

What follows are embodiments and examples of how the platform/application 24 of the present disclosure can utilize AI or machine learning to provide predictions and/or suggestions to the user, based on the user's provided input.

In one embodiment, after the user has entered the requested information including photos of themselves, the platform/application 24 determines the body type and the waist circumference required for longevity and determines the health span for the user.

In one embodiment, the platform/application 24 determines how the user will appear in the future, using (a) a normal aging process, using (b) an accelerated aging process if the suggested protocol is not followed, and using (c) a decelerated aging process if the user follows the suggested protocol.

In one embodiment, the platform/application 24 determines/predicts the user's facial features over a lifetime using normal aging, accelerated aging and decelerated aging.

In one embodiment, the platform/application 24 determines facial features for longevity and determines the user's health span based on input information provided by the user including photos and/or videos of the user taken on the user's electronic device and uploaded to the platform/application 24.

In one embodiment, the platform/application 24 determines the user's rate of aging from provided data (before they begin using the platform/application), accelerated aging (for example, if the user's lifestyle health habits are below average) and decelerated aging (if the user follows the suggested protocols). This will determine their predicted future appearance. In one embodiment, the decelerated aging rate can change depending on their adherence to the suggested protocols.

In one embodiment, the platform/application 24 determines CT Scan measurements for longevity and health span determinations from uploads into the platform/application. For example, CT and MRI measurements are determined by radiology tools available within every Picture Archiving and Communication System (PACS) including measurement of various tissue densities on the CT. Further, the user's level of stress can be estimated from the CT by measuring the volume of adrenal glands as well as other factors.

In one embodiment, the platform/application 24 determines MRI Scan measurements for longevity and health span determinations from uploads into the platform/application.

In one embodiment, the platform/application 24 takes the user's inputted data and combines it with other clinical (non-imaging) data including, for example, epigenetic testing, genetic testing, labs including cancer screening tests, microbiome testing, etc., to provide accurate longevity, rate of aging, and health span determinations.

In one embodiment, the platform/application 24 determines accuracy interval and/or error margins of predicted longevity/health span determination from available information. For example, the accuracy for a photo upload only maybe only 65%, but if the platform/application receives all of the requested user information, the accuracy may be much higher, i.e., 99%.

In one embodiment, the platform/application 24 provides personalized recommendations and suggestions for such thing as specific diet, exercise, lifestyle habits (sleep, stress relief, social connections etc.), vitamins, supplements, medications and any other treatments, etc.

What follows is an exemplary “walk-through” of the Login/Registration process for the user that is presented with the lifestyle system 20 of the present disclosure and the information that is calculated by platform/application 24 and presented to the user as an output on the user's electronic device.

Upon accessing the lifestyle system 20 of the present disclosure, the user may be presented, on the screen of their electronic device, with a Login/Registration screen, such as the exemplary one below:
Login/Register
By clicking register, the system will collect the following information:
Page 1 of registration (Tell us about yourself)
Your name *
Your email * (will be your username)
Your phone
Your dob *
Your gender (radio button: Male/Female)
Your weight and height *
Password/Repeat password *
Button “Next”
Reason(s) you came here today *
DROPDOWN (CAN CHECK MORE THAN ONE)
LONGEVITY EVALUATION
DETECTION OF CONDITIONS I AM NOT BE AWARE ABOUT
WEIGHT LOSS

The user then may be presented with a request for the user to take and upload a picture of themselves, for example:
To provide a better service, we kindly request that you take a selfie of your body (without legs) and face straight and in profile (4 photos total). This is not mandatory but our artificial intelligence will be able to analyze you more accurately to provide much more accurate information. (text needs to be adjusted)
Take a picture of your body front to back
Take a picture of your body in profile (side view)
Take a picture of your face front to back
Take a picture of your face in profile (side view)
Button “Next”

The user may then be presented with a screen that asks them to provide information about their medical history, such as, for example:
Describe your health
Describe your family history
Do you smoke? * (yes/no)
Do you drink? * (yes/no)
Do you Exercise? * (yes/no)

Once the user has entered all or some of the requested information, and activates a button labeled, for example, “Get FREE evaluation of your body,” platform/application 24 will store this information into the database, and will display a page “My Health evaluation”.

On this page, lifestyle system 20 will display on the user's electronic device, an uploaded, AI-generated full image body, where, in one embodiment, there will be a noticeable change so that the user looks slimmer, better and/or younger (based on longevity algorithms used by platform/application 24). In one embodiment, the user will be able to see how their face and body will look in the future (for example, the next 10 years, or 20 years, or next 30 years). The user will be presented with a future image of their face and/or body, depending on an average rate of aging, an accelerated rate of aging (if they continue their current lifestyle) or a slower rate of aging (if they follow the recommendations presented by platform/application 24).

Lifestyle system 20 will then calculate and determine the user's expected healthspan, i.e., the number of predicted healthy years the user can enjoy.

Lifestyle system 20 can then, based on the user's personal and family history, and if they follow the program suggested, determine that the user should be able to eliminate or reverse one or more conditions, such as, for example, obesity, diabetes, coronary arterial disease, etc.

Lifestyle system 20 can also provide personalized recommendations to the user on how they can alter, or maintain their lifestyle, including recommendations such as recommendations related to diet, exercise (e.g., cardio and weight training), sleep and stress management, social connections, cognitive engagement, use of supplements and/or vitamins, medications, etc.

If the user uploads additional information such as, for example, x-rays, or CAT scans, body mass index (BMI), waist circumference, family history of illnesses, etc., lifestyle system 20 can provide more accurate assessments and/or predictions.

Using AI and/or machine learning, lifestyle system 20 is configured to detect a person's silhouette in photos, analyze the image and predict main body parameters, such as, for example, bust, waist, hips, and determine the body type of the user. In one embodiment, lifestyle system 20, via hardware 26, performs the following:
Collects a set of data;
Develops a measurement estimation model;
Integrates a prediction pipeline via, for example, an application program interface; (API) such as by using FastAPI endpoints;
Utilizes rule-based body type classification logic;
Implements image preprocessing & silhouette segmentation using computer vision techniques and machine learning, such as, for example MediaPipe Pose, etc.;
Performs evaluation on different data sources (i.e., the collected dataset+online samples); and
Conducts experiments with model configurations and feature inputs.

### Dataset Analysis and Set Structure

In some embodiments, the final data could be, for example, 200 samples (this number is an example only), which are used for testing and evaluating the quality of the model. Of these, a portion of the samples could be samples obtained from various internet sources. The data collected from the user could include, for example: user photographs with height/weight/waist parameters; data with manually taken measurements; images with different body types and poses, and natural variability, such as, for example, various light conditions, backgrounds, i.e. real world conditions. The information taken from the internet or other sources could, for example, help expand the representativeness of the obtained data set; be used for additional verification of model generalization; include high quality/ideal conditions, i.e., form-fitting clothing, neutral backgrounds, correct pose, etc.

In one embodiment, lifestyle system 20 automatically extracts body measurements from uploaded user images and generates three core outputs: Bust, waist, and hips. For example, when prompted, the user uploads one or more photos of themselves, including front and side views. Lifestyle system 20 detects the silhouette of the user, and analyzes body shape features. Lifestyle system 20 then estimates measurements, i.e., similar to using a tape measure but fully automated.

### Rule-Based Body Type Classification

After predicting bust, waist and hip measurements, lifestyle system 20 applies rule-based logic to determine the user's body type. The classification is based on ratios between the three measurements (i.e., bust, waist, hips).

### Gender-Based Category

Female→Hourglass (balanced bust & hips, narrow waist)
Male→V-Shape (broader upper body relative to waist/hips)

### General Categories (Applied for Both Genders)

Rectangle—similar proportions between bust/waist/hips
Pear (Triangle)—wider hips compared to bust
Apple/Inverted Triangle—larger upper body/waist area relative to lower body

The logic uses proportional measurements rather than only absolute values, ensuring an interpretable and controllable decision process suitable for sizing and recommendation systems.

Thus, lifestyle system 20 successfully implements a full pipeline from image processing to body measurement estimation including:
Developing a front/side image preprocessing pipeline;
Building a model for predicting Bust/Waist/Hips measurements;
Implementing a rule-based body type classification system;
Collecting a dataset and performed an initial quality evaluation;
Demonstrating real examples on test images; and
Achieving measurement results closely aligned with ground truth.

In some embodiments, lifestyle system 20 provides an Optimal Fitness via Epigenetic Enhancement (OFEE) score (also referred to herein as “healthspan score”). An OFEE score is a modified rate of aging taking into consideration the user's genetic factors, current state of health, lifestyle and constant epigenetic changes. Achieving optimal health and fitness involves a synergistic blend of different types of training (e.g., High-Intensity Interval Training (HIIT), resistance training, and aerobic exercise), coupled with smart nutrition (for example, a healthy Mediterranean-style diet, rich in antioxidants etc.), sleep/stress management and personalized insights/enhancements derived from epigenetic testing, all aimed at positively shifting gene expression for better metabolism, muscle growth, health span and longevity, essentially turning lifestyle into potent biological signals.

In one embodiment, lifestyle system 20 is configured to provide an OFEE program, which is a personalized optimal fitness program for each individual with the goal of achieving a state where the user's body functions effortlessly, allowing them to handle daily demands, enjoy activities, and recover quickly, focusing on balanced physical and mental well-being rather than just peak performance or aesthetics. It involves a holistic approach incorporating good diet, supplements, exercise, hydration, sleep, and stress management among other factors. Exercise involved all-around fitness (cardio, strength, flexibility) and adapting to life's needs, feeling energetic, pain-free, and capable of performing various tasks with enthusiasm and ease.

Aspects of the program related to optimal fitness, such as:
Ready state: The ability to adapt, perform when needed, and bounce back quickly, living pain-free.
Function over Form: Feeling free and capable (e.g., hiking, playing, lifting weights) rather than just looking a certain way (e.g., bodybuilder).
Integrated Body: Training the body as a whole system with fundamental movements (e.g., squats, presses) for long-term health, avoiding imbalances from over-specialization.
Balance and consistency: A balanced routine including aerobic activity (e.g., 150+ mins/week), strength training (e.g., 2×/week), good nutrition, sleep, and mental health, etc.
Personalized goals: What's optimal for one person (e.g., a senior) differs from another (e.g., a young athlete), but it should meet the user's life's demands.

Holistic approach: Incorporating good diet, hydration, sleep, and stress management.

### Lifestyle Factors (Nutri(epi)genomics)

Avoidance of harmful external factors such as smoking, excessive alcohol consumption, pollution, radiation, etc.
Nutrition: Diets rich in whole foods, fruits, vegetables (Mediterranean/DASH styles) provide antioxidants and nutrients that support DNA methylation and gene expression.
Stress and Sleep Management: Good Sleep, Stress relief, mindfulness can regulate DNA methylation and reduce inflammation, positively impacting gene expression.
Social connections
Access to healthcare
Aerobic Exercise including HIIT (High-Intensity Interval Training): Short bursts of intense effort boost mitochondrial function, fat metabolism, and cardiovascular health by influencing epigenetic marks. Supports overall cellular function, repair, and metabolic health, contributing to epigenetic rejuvenation.
Resistance Training (Strength Training): Builds muscle, improves insulin sensitivity, and alters DNA methylation in muscle growth genes, enhancing body composition.

### Advanced Approaches

Epigenetic Testing: Provides personalized insights into your genetic predispositions, allowing for tailored fitness and nutrition plans.
Diagnostic Imaging (MRI, CT, etc.), microbiome, labs, etc.
AI & Wearables: Can create dynamic fitness plans based on real-time biometric data, enhancing consistency and adaptation.

By combining some or all of the above elements, a powerful environmental pressure is created that signals the user's genes to support metabolic function, cellular repair, and muscle adaptation, leading to improved health, enhanced performance, and potentially slowing epigenetic aging.

### OFEE Score Algorithm

In some embodiments, lifestyle system 20 calculates an OFEE score (OS), which is a modified rate of aging score, i.e., the speed at which an individual experiences age-related physical and physiological decline. This is distinct from chronological aging, which is simply the passage of time in years. OS varies significantly between individuals, influenced by (epi)genetics, lifestyle, and environment, but generally involves biological changes accelerating in mid-life (around 45-60 years of age), measured by biomarkers like DNA methylation and telomere length, showing some people age faster while others age slower than their chronological age.

Aging accelerates after 40, not gradually, but in significant bursts around age 44 and again at 60, impacting metabolism, muscle, skin, immune function, and cardiovascular health, with the 40s showing shifts in fat/alcohol processing and the 60s a major immune decline, making diseases like Alzheimer's and heart issues more likely as the body's molecular balance shifts dramatically.

Around Age 44 (mid-40s)
Metabolism Slows: Metabolism begins to slow, making weight gain easier, and fat distribution changes.
Skin & Muscle: Collagen production decreases, leading to sagging skin and wrinkles; muscle mass starts to decline.
Digestion: The body becomes less efficient at breaking down fats, alcohol, and caffeine.
Cardiovascular: Risk for cardiovascular issues starts increasing.
   Around Age 60 (early 60s)
Immune System: A significant drop in immune function occurs, increasing inflammation. Cognitive/Neurological: Neurological and cognitive processes can slow down.
Hormones: Lower estrogen (women) and testosterone (men) impact mood, bone health, and strength.
Disease Risk: Sharper increases in risks for Alzheimer's, Parkinson's, osteoporosis, and hypertension are observed.

OS rate of aging isn't a smooth, linear decline but rather two major “waves” of change, according to research tracking thousands of molecules. Recognizing these points helps focus preventive strategies (diet, exercise, check-ups) to manage these transitions and extend healthy life.
Molecular Tipping Points: Mid 40s and early 60s represent a key period where biological changes accumulate, leading to more noticeable health issues like vision/hearing loss or increased disease risk.
Critical Health Window: This burst highlights the 40s and 60s as crucial times for proactive health monitoring and intervention.

Factors that influence OS rate of aging:
Individual rates of aging depend on key factors including overall health condition, diet, exercise, stress level, sleep, and overall health, with healthy habits potentially adding health span.

### Individual Aging Rate

Biological vs. Chronological:
Biological age (how old a body functions) can differ from your chronological age (years since birth).

### Varying Speeds:

Some individuals age biologically at twice the rate of their chronological age, while others age much slower, maintaining a younger biological state.

### Key Acceleration:

The aging process appears to significantly speed up between ages 45 and 55, with specific molecular shifts noted around 44 and 60.

### Measurements:

Researchers use biological clocks based on DNA methylation patterns or telomere length to quantify aging rate, with faster aging linked to increased disease and mortality risk.

### Factors Influencing Aging Rate

Genetics: Partly determines your inherent aging pace.
Lifestyle: Diet (fruits, veggies), exercise, sleep, avoiding smoking/alcohol, and managing blood pressure/sugar significantly impact rate.

Biological Decline: Aging at a biological level involves the accumulation of molecular and cellular damage over time, leading to a gradual decrease in physical and mental capacity.

Variability: The rate of aging is highly variable among individuals. Some people age faster biologically than others of the same chronological age.

Health and Mortality: A faster rate of aging is associated with an earlier onset of chronic diseases, increased vulnerability to disability, and a higher mortality risk.

### OFEE Score (OS) Rate of Aging Algorithm Calculation

OS is the ratio of biological age to chronological age. It is assumed that every user has an intrinsic OS score of 1.0, so each user is essentially competing only with themselves. The difference is the variability below or above 1.0 for everyone.
OS<1 suggests an individual is aging slower than expected.
OS>1 suggests an individual is aging faster than expected.
Modifiable: OS can be influenced by lifestyle and environmental factors, such as diet, supplements, certain medications, exercise, sleep patterns and stress relief, which suggests it may be modifiable through interventions.

### Factors Influencing OS:

Genetic/fixed factors-male/female, age, family history, etc.
Current patients' health (medical conditions, etc.)
External factors-diet, lifestyle, etc.

### Epigenetic Factors:

Genetics: Partly determines your inherent OS rate of aging
Lifestyle: Diet (fruits, veggies), exercise, sleep, avoiding smoking/alcohol, and managing blood pressure/sugar significantly impact rate.
Environment: Exposure to toxins and stress can accelerate aging.

### Confirmation:

DNA methylation patterns or telomere length to quantify aging rate, with faster aging linked to increased disease and mortality risk.

### OS Calculations

Changes with age—significant acceleration after age 45 and again additional acceleration after age 60
OFEE Score-(adjusted rate of aging)
Ranges from 0.67 to 1.33
Depending on various variables discussed below, some users will be able to achieve an OS score as low as 0.67, but not everyone is able to do so. The same is true in the opposite direction. This range is determined by several factors including current chronological age and other fixed factors, genetics.

### EXAMPLES

It is assumed that every user has an intrinsic OS score of 1.0, so each user is essentially competing only with themselves. An average person with average life expectancy has an OFEE score of 1.0, by definition. This means that most individuals with average genetics, lifestyles, external factors, etc. will age 1 year biologically for every year lived chronologically and expect to have a standard life expectancy.

Note: For individuals >30 years old, OS calculations will begin from age 30. In the example below for an individual who is 60, adjustments will start from age 30. For individuals with an OS score <1.0, adjustments will continue beyond the life expectancy assuming potential longevity until about 120 years of age. For individuals with an OS score >1.0, adjustments will continue below the life expectancy.

### Extreme Examples

Individuals<45 years old, and completely healthy and following the program suggested by lifestyle system 20 can approach an OFEE score of 0.667. This means that every year this person lived chronologically he/she ages only 8 months biologically (i.e., slowed aging). By utilizing various epigenetic enhancement procedures and therapies, reverse aging (a person's biological age becomes “younger”) with every chronological years) may be possible.

A 30-year-old individual, in this example, will age a lot slower and will have a biological age as well as physical appearance 10 years younger at age 60 (biological age of 50) compared to the general population. The user's health span can also be estimated to be 10 years in this example. This same individual will have a biological age as well as appearance 20 years younger at age 90 (biological age of 70) compared to the general population. Health span can also be estimated to be 20 years in this example. This same individual will have a biological age as well as appearance 30 years younger at age 120 (biological age of 90) compared to the general population. Health span can also be estimated to be 30 years in this example.

Individuals>60 years old with very unhealthy lifestyles, poor genetic and external factors etc., can approach an OFEE score of 1.333. This means that every year this person lived chronologically he/she ages 1 year and 4 months biologically (accelerated aging).

A 60-year-old individual in this example will age much faster and will have a biological age as well as appearance 10 years older at age 60 (biological age of 70) compared to the general population. This individual will unlikely reach a standard life expectancy and calculations will continue until biological age=life expectancy (unless the OS is adjusted).

In some embodiments, lifestyle system 20 may use weighting factors such as the ones discussed below, to determine a user's rate of aging, i.e., an OFEE score, based on user input. The calculations may use machine learning and/or AI, but are not limited to their use.

### EXAMPLES

Each of the following factors have a weight of 0.25+/−0.0625 bonus or penalty adjustment
1. Genetic/fixed factors-male/female, age, family history etc. These factors are discussed above. As also previously discussed herein, there are adjustments calculated due to molecular tipping points at ages 45 and 60. In one embodiment, an age 45 penalty adjustment of 0.0125 is used and at age 60 an additional penalty adjustment of 0.0250 is used.
2. Current patient's health (medical conditions, etc.). As discussed above, for example, a completely healthy user acquires a full bonus, while a user having multiple serious medical conditions acquire a full penalty.
3. External factors/lifestyle-diet, sleep, stress relief, etc., are incorporated. In some examples, as previously discussed:
Diet 0.3
Smoking/alcohol/external toxins 0.15
Exercise 0.15
Sleep 0.1
Stress management 0.1
Social connection 0.1
Access to Healthcare 0.1
   Example: A user follows a “healthy lifestyle” 100% of the time and receives a full bonus. If the user does not follow a healthy lifestyle in any of the categories—a full penalty is applied.
4. Epigenetic enhancements
The user undergoes regular screenings and tests (diagnostic imaging, epigenetic, microbiome, labs).
The user takes supplements
The user takes longevity medications—e.g., metformin, GLP1, SGLT2 inhibitors, Rapamycin, etc.

Example: the user follows the suggested program completely—a full bonus is applied. If the user does not follow the suggested program—a full penalty is applied. If, for example, the user follows some of the program suggestions and not others, various weighting factors are applied.

In some embodiments, the OS constantly changes and will be recalculated periodically (for example, every three months) based on lifestyle and epigenetic changes and measurements.

There are numerous adjustments factors given for any changes in the lifestyle and epigenetic changes, including (for example):
Discovery or resolution of health conditions (diabetes, gout, etc.)
Changes in diet and nutrition
OFEE AI measurements (body and face) and resultant crucial information such as body shape, fat %, WHR (waist to hip ratio) measurement, wBMI (waist adjusted BMI), etc.
Changes in exercise/fitness (as measured as VO2 Max, muscle quality/density, grip strength, etc.
Addition of certain medications or supplements
Medical imaging findings (typically every 1-2 years)—fatty liver, coronary calcifications, bone and muscle density, etc.
Changes in other testing (every 3 to 12 months)—epigenetic, labs, microbiome etc.
External factors/lifestyle modifications-sleep, stress relief, social connections, etc.
Certain epigenetic procedures (stem cells, etc.)

In some embodiments, OS changes will be tracked every quarter and individuals will be rewarded or punished (“carrots and sticks”)=gamifications. For example, an individual who was able to change their OS from 0.9 to 0.8 will be given certain points and rewards. Individuals who can accelerate these changes will be given special additional rewards. For example, going from an OS of 1 to an OS 0.97 and from an OS of 0.97 to an OS of 0.9 in the 2nd quarter represents significant acceleration (exponential). This progress or acceleration will count more than just OS on its own, especially for individuals who start with OS>1.

In some embodiments, the OS also dictates the visual appearance of individual over the years within and individuals can project their appearance via photographs/videos (both face and body) at specific intervals, e.g., every 3 months. For example, a 30-year-old individual in the example above can see themselves at the age of 60 looking like an average person at 50 (biological age of 50).

Lifestyle system 20 can also provide other calculations, such as, for example, biological age, which can be calculated from epigenetic tests, and health span (in years, i.e., the number or additional healthy years. Increased healthspan for the user can be calculated from the OS score as discussed herein. Lifestyle system 29 after analyzing all the factors, uses algorithms and logic to compute the maximum number of points that each user can earn (goal or runway) as well as actual points earned with every change to their lifestyle.

In one embodiment, not including bonus points, the goal is earning up to a given number of points (for example, 2,250 points) (not including bonus); where 100 points=1 additional year of longevity added.

This includes the following factors discussed above:
1. Genetic/fixed factors-male/female, age, family history, etc.
2. Current patient's health (medical conditions, etc.)
3. External factors-diet, lifestyle, etc.
4. Epigenetic enhancements

Lifestyle system 20 can obtain reports from third party facilities that can be used, in addition to the information provided by the user, to assist in generating OFEE scores. For example, third party reports might be, for example, a cardiovascular report of the user, which could include such information as the user's cardiothoracic ratio (CTR), heart length, inner chest length, coronary artery calcifications (CAC), thoracic aortic calcifications (TAC), abdominal aortic calcifications (AAS), aortic valve calcification (AVC), aortic annulus calcifications (AAnc), mitral annulus calcifications (MAnC), etc. Lifestyle system 20 can use some or all of this information in conjunction with the user's provided information, to arrive at an OFEE score.

Similarly, lifestyle system 20 can obtain from a third party and use a metabolic report of the user to assist in generating OFEE scores. The metabolic report can include such things as the user's visceral fat area (VFA0, subcutaneous fat area (SFA), visceral to subcutaneous ratio (VSDR0, subcutaneous fat density (SFD), abdominal circumference, liver attenuation (LA), bone attenuation (BA), skeletal muscle area (SMA), and skeletal muscle density (SMD).

The above are merely examples of third party reports that lifestyle system 20 may use, in addition to the information provided by the user, to generate an OFEE score, and to otherwise provide health span and rate of aging predictions and life style changes and recommendations to the user.

It will be appreciated by persons skilled in the art that the present embodiments are not limited to what has been particularly shown and described herein above. In addition, unless mention was made above to the contrary, it should be noted that all of the accompanying drawings are not to scale. A variety of modifications and variations are possible in light of the above teachings.

1. A computer-based method for extending a user's healthspan and decreasing the user's rate of aging, the method comprising:
receiving user information via a lifestyle platform, the user information including identification information, medical information, and at least one of biometric information, lab information, medical imaging reports and images of the user;
processing and analyzing the received information using at least one of AI technology, machine learning and algorithms; and
providing accurate health, wellness and longevity information and recommendations to the user.

2. The computer-based method of claim 1, further comprising using a medical data management system having blockchain technology to secure the received user information.

3. The computer-based method of claim 1, wherein the processing and analyzing step comprises consideration of one or more of genetic and fixed factors, the user's current health status and external factors, including the user's diet and lifestyle.

4. The computer-based method of claim 3, wherein a first healthspan score is determined based upon the genetic and fixed factors and the user's current health status, and wherein the first healthspan score can be increased depending upon the external factors to result in a final healthspan score.

5. The computer-based method of claim 4, wherein the final healthspan score is adjusted depending upon one or more of the sex of the user, at least one of the height and waist-adjusted body mass index (wBMI) of the user, the user's family health history, certain current medical conditions of the user, and a current physical activity level of the user.

6. The computer-based method of claim 1, further comprising integrating the user information with hospital and medical facilities.

7. The computer-based method of claim 1, further comprising determining the user's rate of aging based upon the received user information, the rate of aging being one of an average rate of aging, an accelerated rate of aging and a decelerated rate of aging.

8. The computer-based method of claim 1, further comprising estimating the user's future physical appearance.

9. The computer-based method of claim 1, further comprising receiving, from the user, user-specific goals including at least one of losing a specific amount of weight, eliminating one or more medical conditions, and achieving a desired rate of aging within a specified period of time.

10. The computer-based method of claim 1, further comprising providing an estimate of one or more of the user's physical characteristics and age progression based at least upon the images of the user.

11. The computer-based method of claim 1, further comprising identifying the user's predisposition in developing certain medical conditions, and providing recommendations how to eliminate, avoid and/or limit the certain medical conditions.

12. The computer-based method of claim 7, further comprising utilizing at least one of biometric devices, smart watches and smart glasses to present to the user the accurate health, wellness and longevity information and recommendations and rate of aging determination.

13. The computer-based method of claim 7, wherein the received user information includes current diet, the method further comprising providing to the user a recommended improved diet and using the recommended improved diet to calculate revised health, wellness and longevity information and to provide updated rate of aging information.

14. The computer-based method of claim 1, further comprising providing one or more notifications to the user to remind the user to one or more of access the platform more frequently, maintain the health, wellness and longevity information and recommendations, and provide additional requested information.

15. A computer-based system for extending a user's healthspan and decreasing the user's rate of aging, the system comprising:
processing circuitry including one or more processors, memory and a communication interface,
the communication interface configured to receive user information including identification information, medical information, and at least one of biometric information, lab information, medical imaging reports and images of the user;
the one or more processors configured to process and analyze the received information using at least one of AI technology, machine learning and algorithms; and
the communication interface configured to provide accurate health, wellness and longevity information and recommendations to the user.

16. The computer-based system of claim 15, further comprising a medical data management system having blockchain technology to secure the information provided by the user.

17. The computer-based system of claim 15, wherein the processing and analyzing comprises consideration of one or more of genetic and fixed factors, the user's current health status and external factors, including the user's diet and lifestyle.

18. The computer-based system of claim 17, wherein a first healthspan score is determined based upon the genetic and fixed factors and the user's current health status, and wherein the first healthspan score can be increased depending upon the external factors to result in a final healthspan score.

19. The computer-based system of claim 18, wherein the final healthspan score is adjusted depending upon one or more of the sex of the user, at least one of the height and waist-adjusted body mass index (wBMI) of the user, the user's family health history, certain current medical conditions of the user, and a current physical activity level of the user.

20. The computer-based system of claim 19, wherein the one or more processors are further configured to determine the user's rate of aging based upon the received information, the rate of aging being one of an average rate of aging, an accelerated rate of aging and a decelerated rate of aging.
